(19) United States
(12) Patent Application Publication
(10) Pub. No.: US 2025/0137995 A1
(43) Pub. Date: May 1, 2025

(54) IN VITRO QUALITY SCREENING OF HUMAN CORNEAL STROMAL STEM CELLS FOR CELL-BASED THERAPY OF CORNEAL SCARRING

(71) Applicant: UNIVERSITY OF PITTSBURGH - OF THE COMMONWEALTH SYSTEM OF HIGHER EDUCATION, Pittsburgh, PA (US)

(72) Inventors: Hin-Fai YAM, Pittsburgh, PA (US); Yiqin DU, Pittsburgh, PA (US); Vishal JHANJI, Pittsburgh, PA (US); Isabelle BILLIG, Pittsburgh, PA (US); Jose Alain SAHEL, Pittsburgh, PA (US); Deepinder K. DHALIWAL, Pittsburgh, PA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 19/010,689

(22) Filed: Jan. 6, 2025

Related U.S. Application Data
(63) Continuation of application No. PCT/US2023/027823, filed on Jul. 14, 2023.
(60) Provisional application No. 63/411,494, filed on Sep. 29, 2022.
(60) Provisional application No. 63/389,100, filed on Jul. 14, 2022.

(51) Int. Cl. G01N 33/50; C12N 5/079

(52) U.S. Cl. CPC G01N 33/5023; C12N 5/0621; G01N 33/5055; G01N 33/5073; G01N 2333/4703; G01N 2333/70596; G01N 2333/916; G01N 2333/96413; G01N 2333/96419; G01N 2800/52

(57) ABSTRACT

The present disclosure relates to a method of determining effectiveness of corneal stromal stem cell therapy through evaluation of stem cell markers, inflammation markers, and assessing a scarring index.

### CROSS-REFERENCE TO RELATED APPLICATIONS

This application is a continuation of International Patent Application No. PCT/US2023/027823, filed Jul. 14, 2023, which claims priority to U.S. Provisional Patent Application Ser. No. 63/411,494 filed on Sep. 29, 2022 and U.S. Provisional Patent Application Ser. No. 63/389,100 filed on Jul. 14, 2022, the contents of each and all of which are hereby incorporated by reference in their entireties.

### 1. FIELD OF INVENTION

The present disclosure relates to the novel methods of selecting cultivated donor corneal stromal stem cells (CSSCs) with optimal healing/regenerative capability for administering to patients with corneal scarring.

### 2. BACKGROUND

Corneal blindness is a leading cause of vision loss worldwide (World Report on Vision, WHO 2019; www.who.int/publications/i/item/world-report-on-vision). It is estimated that 35% to 50% of blindness is due to corneal scarring [1]. Conventional treatment includes partial or full-thickness corneal transplantation using healthy donor corneas. Corneal transplantation is one of the most frequently performed solid organ transplant surgeries globally. Despite the advances in techniques of keratoplasty, only 1 in 70 individuals with treatable corneal scarring can undergo this surgery due to a multitude of social and economic issues, and most importantly a limited supply of transplantable donor corneas, especially in the developing countries.

Since the discovery of corneal stromal stem cells (CSSCs) in 2005, stem cell therapy is an attractive approach to prevent or remediate corneal scarring [2-4]. Applying cultivated CSSCs in a fibrin gel to the injured corneal tissue in preclinical animal models can suppress injury-associated inflammation [5], reduce fibrosis, and promote native-like corneal stromal tissue regeneration, leading to reduced corneal opacities/scar formation and better recovery of corneal transparency [6-11]. An interventional clinical trial using corneal stromal mesenchymal stem cell therapy in patients with corneal haze from infection, after laser surgery, or collagen crosslinking, is underway in LV Prasad Eye Institute, India (NCT02948023); the preliminary results have demonstrated safety and effectiveness of corneal scar reduction and vision recovery [6]. However, not every batch of donor-derived CSSCs cultivated in vitro shows suitable anti-scaring and healing activity.

There remains a need in the art to improve and address variations in CSSC effectiveness due to stem cell stability and other measurable cell features. The present disclosure addresses this need.

### 3. SUMMARY

The present disclosure provides methods for determining anti-scarring potential of corneal stromal stem cells (CSSC) s when used for cell therapy in patients with corneal scarring disease.

In certain non-limiting embodiments, the method of determining effectiveness of CSSC therapy comprises obtaining CSSC sample; measuring expression of at least one stemness marker in the CSSC sample for stem cell stability (ΔCT); creating a conditioned medium by culturing the CSSC sample until the cell culture medium contains the desired level of extracellular product; introducing the CSSC sample conditioned medium into a cell-based model of inflammation; measuring the expression of at least one biomarker in the cell-based model (ΔCT); determining a ratio of inflammation of the CSSC sample (RInflam), wherein the ratio of inflammation is based on the expression fold change of at least one biomarker in the cell-based model; and determining a scarring index (SI) score of the CSSC sample, wherein the scarring index is based on the expression of at least one stemness marker in the CSSC in (c) and the expression of at least one biomarker in the cell-based model (e) wherein an SI score is representative of the effectiveness of the CSSC sample for reduction of corneal scar formation in CSSC therapy.

In certain embodiments, the at least one stemness marker is selected from the group consisting of ABCG2, NESTIN, and combinations thereof.

In certain embodiments, the ratio of inflammation is determined by an osteoclastogenesis assay and calculating a sum of expression fold change ratio of targeted osteoclast genes. In certain embodiments, the targeted osteoclast genes comprise alkaline phosphatase 5 [ACP5], matrix metalloproteinase 9 [MMP9], and cathepsin K [CTSK], and the ratio of inflammation is calculated according to:

ΣRInflam_{ACP5+MMP9+CTSK}=ACP5(naïve/denatured)+MMP9(naïve/denatured)+CTSK(naïve/denatured)

In certain embodiments of the method, the scarring index score is calculated according to:

$$Scarring Index ( S I ) = [ {2}^{Δ CT ( ABCG 2 )} + {2}^{Δ CT ( NES )} ] / m + {2}^{\sum RInflam} / n$$

wherein m and n represent constants.

According to the presently disclosed method, an SI score of less than 10 represents effectiveness of the CSSC sample for reduction of corneal scar formation in CSSC therapy. In certain embodiments, the reduction of corneal scar formation is about 50%.

In other embodiments, an SI score greater than 10 represents reduced effectiveness of the CSSC sample for reduction of corneal scar formation in CSSC therapy.

In certain embodiments the expression of at least one stemness marker or osteoclast gene is determined by reverse transcription polymerase chain reaction.

In certain embodiments the cell-based model of inflammation comprises a cell of the hematopoietic lineage capable of undergoing osteoclastogenesis. In certain embodiments, the cell-based model comprises a macrophage cell. In certain embodiments, the macrophage cell comprises a RAW264.7 cell or derivative thereof. In certain embodiments, the RAW cell or derivative thereof is induced to undergo osteoclast differentiation. In certain embodiments, the RAW cell is pre-treated with naïve or heat-denatured conditioned media prior to induction of osteoclast differentiation.

In certain embodiments, the effectiveness of CSSC therapy is determined in an in vivo model of corneal stromal injury. In certain embodiments, the in vivo model of corneal injury is a murine mouse model of anterior corneal stromal injury.

The disclosed subject matter further provides a method of treating, reducing, or preventing corneal scarring in a subject in need thereof. In certain embodiments, the method comprises obtaining a corneal stromal stem cell (CSSC) sample, determining a scarring index (SI) score of the CSSC sample, wherein an SI score is representative of the effectiveness of the CSSC sample as a therapy for treating, reducing or preventing of corneal scar formation in a subject; formulating an anti-scarring therapy comprising the CSSC sample, and administering a therapeutically effective amount of the anti-scarring therapy to the subject.

### 4. BRIEF DESCRIPTION OF THE DRAWINGS

For a more complete understanding of the present disclosure and its features and advantages, reference is now made to the following description, taken in conjunction with the accompanying drawings.

FIGS. 1A-1B illustrates relative RNA abundance of stem cell markers among human CSSC batches from different donors. ACT represents the difference of Cycle Threshold (CT) of gene of interest against CT of housekeeping 18s of the sample. FIG. 1A shows passage-dependent changes of stemness genes in 3 representative CSSC batches derived from different donors. From P1 to P3, the cellular expression of ABCG2 and NESTIN remained consistent whereas Pax6, Bmi1, Sox2, Oct4 and NGFR gradually downregulated. FIG. 1B shows the expression patterns of ABCG2 and NESTIN were mostly consistent among 18 donor CSSC batches at P2.

FIG. 2 illustrates the anti-inflammatory property of donor CSSC (n=18 batches) at P2. Mouse RAW264.7 cells pre-incubated with heat-denatured CMconc showed upregulated osteoclast gene expression (ACP5, MMP9, and CTSK) (blue-colored columns) after RANKL/ConA induction, similar to the control without CMconc treatment (black). All 3 genes were downregulated after treatments using native CMconc (orange) from most CSSC batches, but not samples from HC515, 534, and 572 (in bracket). The treatment with CMconc from human stromal fibroblasts (SF) serves as a negative control and it showed poor anti-inflammatory effect.

FIG. 3 illustrates human CSSC treatment using a mouse model of anterior corneal stromal scarring. At day 14 post-treatment, mouse corneas showed different degrees of scarring with reference to naïve and untreated wound controls (WND). Most CSSC batches showed different degrees of anti-scarring effect, but some were ineffective to prevent scarring (HC515, 572 and 534).

FIG. 4 illustrates the correlation of in vivo CSSC treatment outcome (% scarring) with in vitro SI calculated by the invented formula predicting the anti-scarring potency of CSSC. FIG. 4A shows the distribution of SI (round dots) calculated for each donor CSSC batch (n=18). Labels on the x-axis indicate CSSC batches from different donors. CSSCs with SI<10 are blue-colored dots whereas cells with SI>10 are orange-colored. FIG. 4B shows the mean percentages of scarring area (squares) detected in mouse corneas after treatment with CSSC batches arranged with the same order as in A (n=8 mice per cell treatment). Most CSSC treatments resulted in scar reduction, when compared with the untreated wound control (100%, dark horizontal line), except HC572 and 515 with ineffective outcomes. Treatment with CSSC batches having in vitro SI<10 showed consistent scar inhibition (marked by the blue squares), with an average of 32.7±17.4% scar inhibition. The blue dotted line is a stable regression line for cells with SI<10 (A) and their treatment outcome (% scarring), and the correlation was consistent among these cell batches. For CSSC having in vitro SI>10, the treatment outcomes showed moderate to ineffective scar inhibition. The regression line indicated an increasing trend of scar formation (orange dotted line). The treatment outcomes by these 2 groups of cells: SI<10 (blue) versus SI>10 (orange) showed a statistically significant difference (P=0.0312, Wilcoxon Signed Rank test).

FIG. 5 depicts mouse corneal images showing the treatment outcomes with different donor CSSC batches with their respective SI values indicated. The results were compared with naïve and untreated wound controls. The treatment with donor stromal fibroblasts serves as a negative control of scar inhibition. Donor CSSC with SI<10 resulted in scar inhibition, whereas cells with higher SI values (SI>10) showed moderate to ineffective scar prevention. Corneas treated with HC515 and HC572 showed similar scar manifestation as the wound control.

### 5. DETAILED DESCRIPTION

The present disclosure relates to methods of determining the effectiveness of corneal stromal stem cells (CSSC) in the treatment of corneal scar reduction and healing activity. The present disclosure is based, in part, on the discovery that variations in CSSC effectiveness are attributable to stem cell stability. Using the methods disclosed herein, the determination of effectiveness can be calculated by a scarring index provided below.

For purposes of clarity of disclosure, but not by way of limitation, the detailed description of the presently disclosed subject matter is divided into the following subsections:
5.1 Definitions; and
5.2 Methods.

### 5.1 DEFINITIONS

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of”, and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

An “effective amount” or “therapeutically effective amount” is an amount effective, at dosages and for periods of time necessary, that produces a desired effect, e.g., the desired therapeutic or prophylactic result. In certain embodiments, an effective amount can be formulated and/or administered in a single dose. In certain embodiments, an effective amount can be formulated and/or administered in a plurality of doses, for example, as part of a dosing regimen.

As used herein, the terms “expression” or “expresses,” as used herein, refer to transcription and translation occurring within a cell, e.g., mammalian cell. In certain embodiments, the level of expression of a gene and/or nucleic acid in a cell can be determined on the basis of either the amount of corresponding mRNA that is present in the cell or the amount of the protein encoded by the gene and/or nucleic acid that is produced by the cell. For example, mRNA transcribed from a gene and/or nucleic acid is desirably quantitated by northern hybridization. Sambrook et al., Molecular Cloning: A Laboratory Manual, pp. 7.3-7.57 (Cold Spring Harbor Laboratory Press, 1989). Protein encoded by a gene and/or nucleic acid can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

The term “gene expression” is used in the broadest sense, and includes methods of quantification of mRNA and/or protein levels in a biological sample.

As used herein, the term “corneal scarring” refers to any opacity or irregularity on or within the corneal surface that can compromise its ability to transmit and reflect light correctly. In certain embodiments, corneal scarring impairs vision. In certain embodiments, corneal scarring in the central cornea impairs vision.

The term “nucleic acid molecule” and “nucleotide sequence,” as used herein, refers to a single or double-stranded covalently-linked sequence of nucleotides in which the 3′ and 5′ ends on each nucleotide are joined by phosphodiester bonds. The nucleic acid molecule can include deoxyribonucleotide bases or ribonucleotide bases, and can be manufactured synthetically in vitro or isolated from natural sources.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing scarring, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the terms “prevent,” “preventing,” or “prevention,” “prophylactic treatment,” and the like refer to reducing the probability of developing a disorder or condition in a subject who does not have but is at risk of or susceptible to developing a disorder or condition. The prevention can be complete (i.e., no detectable symptoms) or partial so that fewer symptoms are observed than would likely occur absent treatment. The terms further include a prophylactic benefit. For disease or condition to be prevented, the compositions can be administered to a patient at risk of developing a particular disease or to a patient reporting one or more of the physiological symptoms of a disease, even though a diagnosis of this disease cannot have been made.

The term “reduction of corneal scar formation”, “reduced scar formation”, or “scar reduction” as used herein refers to any tissue response that reflects an improvement in wound healing. Specifically, improvement in conditions such as, but not limited to, hyperplasia or adverse reactions to post-cellular trauma are contemplated. It is not contemplated that all scar tissue must be avoided. It is enough if the amount of scarring or hyperplasia is reduced as compared to untreated patients.

The term “wound” as used herein, denotes a bodily injury with disruption of the normal integrity of tissue structures. In one sense, the term is intended to encompass a “surgical site”.

In another sense, the term is intended to encompass wounds including, but not limited to, contused wounds, incised wounds, lacerated wounds, non-penetrating wounds (wounds in which there is injury to underlying structures), open wounds, penetrating wound, perforating wounds, puncture wounds etc.

As used herein, the term “donor” refers to any organism that is the source of cells or tissue. As used herein, the term “cell” refers to any suitable cell for use in the present disclosure, e.g., eukaryotic cells. For example, but not by way of limitation, suitable eukaryotic cells include animal cells, e.g., mammalian cells. In certain embodiments, suitable cells are cultured cells. In certain embodiments, suitable cells are cell lines obtained or derived from mammalian tissues which are able to grow and survive when placed in media containing appropriate nutrients and/or growth factors.

As used herein, the term “corneal stromal stem cells (CSSC)” refers to the population of stem cells which reside in the corneal stromal niche.

As used herein, the term “stem cell” includes any stem or progenitor cell, whether from a human or non-human source, and cells derived from stem cells that retain characteristics of progenitor cells.

As used herein, the term “culture” refers to contacting a cell or tissue with a cell or tissue culture medium under conditions suitable to the survival, growth and/or proliferation of the cell or tissue.

As used herein, the term “conditioned medium” refers to a cell culture medium that contains components that were not present in the starting cell culture medium that is used to culture and feed the cells, but is produced by the cultured cells and enters the medium. Also within the meaning of the term conditioned medium is a cell culture medium that contains components that are initially present in the pre-conditioned medium, but whose concentration is increased during the culture process. Also within the meaning of the term “conditioned medium” is medium in which corneal CSSCs are grown. The CSSC conditioned medium as described herein is obtained by culturing of CSSC in a manner known in the art.

The term “predict” refers to a forecast or calculation of the effectiveness of the CSSC therapy, and healing/regenerative capability for the treatment of corneal scarring in patients. Prediction generally refers to a forecast of the probable course or outcome of the CSSC therapy in treating corneal scarring in a patient. Prediction can use the information of the individual as well as external data to compare against the information of the individual, such as population data, response rate for patients, family or other genetic information, and the like. As a general concept, prediction markers screened for this purpose are preferably derived from sample data according to the therapy to be predicted.

As used herein the term “batch” refers to a technique, i.e., a mode of manufacturing cells or tissue such as CSSCs, in which the CSSCs in question are produced stage by stage over a series of unit operations. All of the material that is to be processed passes a given unit operation before any of said material is processed in the subsequent unit operation.

### 5.2 METHODS

Vision loss due to corneal blindness requires corneal transplantation using healthy donor corneas. However, treatment is restricted by a limited supply of transplantable donor corneas. While corneal stromal stem cells (CSSCs) provide an alternative stem cell therapy approach to prevent or remediate corneal scarring, not every batch of donor-derived CSSCs shows suitable regenerative activity. The present disclosure provides methods for determining the effectiveness of CSSC therapy. For example, but not by way of limitation, the methods disclosed herein can be used to determine stem cell stability and obtain measurable qualities of cell features. The methods disclosed herein provide quality control standards to select CSSCs with optimal healing/regenerative capability (high Corneal Regenerative Potency) for administering to patients with corneal scarring.

In certain embodiments, methods of the present disclosure include obtaining a CSSC sample, measuring expression of at least one stemness marker in the CSSC sample for stem cell stability, determining a ratio of inflammation of the CSSC sample, and determining a scarring index (SI) score of the CSSC sample, wherein the SI score is representative of the effectiveness of the CSSC sample for reduction of corneal scar formation in CSSC therapy. The methods disclosed herein allow an unexpected and significant improvement of determining the effectiveness of CSSC therapy.

In certain embodiments, the present disclosure includes obtaining a CSSC sample. In certain non-limiting embodiments, a CSSC sample includes, but is not limited to, cells in culture, cell supernatants, cell lysates, serum, blood plasma, biological fluid (e.g., blood, plasma, serum, stool, urine, lymphatic fluid, ascites, ductal lavage, saliva and cerebrospinal fluid) and tissue samples. The source of the CSSC sample may be solid tissue (e.g., from fresh, frozen, and/or preserved organ, tissue sample, biopsy, or aspirate), blood or any blood constituents, bodily fluids (such as, e.g., urine, lymph, cerebral spinal fluid, amniotic fluid, peritoneal fluid or interstitial fluid), or cells. In certain non-limiting embodiments, the sample source is cornea tissue. In certain embodiments, the sample source may be obtained from a “biopsy sample” or “clinical sample,” which are samples derived from a subject. In certain embodiments, the biopsy sample is obtained from the transitional region between optically clear cornea and opaque sclera, known as limbus. In certain embodiments, biopsy samples consists of a population of limbal epithelial stem cells and mesenchymal cell. In certain embodiments, the sample source includes one or more corneal cells from a subject.

In certain embodiments, the present disclosure includes measuring the expression of at least one stemness marker for stem cell stability in a CSSC sample.

In certain non-limiting embodiments, the stemness marker is measured by nucleic acid hybridization analysis.

In certain non-limiting embodiments, the stemness marker is measured by DNA hybridization, such as, but not limited to, Southern blot analysis.

In certain non-limiting embodiments, the stemness marker is measured by RNA hybridization, such as, but not limited to, Northern blot analysis. In certain embodiments, Northern blot analysis can be used for the detection of a stemness marker, where an isolated RNA sample is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography to detect the presence of a fusion gene in the RNA sample.

In certain non-limiting embodiments, the stemness marker is detected by nucleic acid sequencing analysis.

In certain non-limiting embodiments, the stemness marker is detected by probes present on a DNA array, chip or a microarray. For example, and not by way of limitation, oligonucleotides corresponding to at least one stemness marker can be immobilized on a chip which is then hybridized with labeled nucleic acids of a sample obtained from a subject. Positive hybridization signal is obtained with the sample containing the stemness marker transcripts.

In certain non-limiting embodiments, the expression of at least one stemness marker is measured by a method comprising Reverse Transcription Polymerase Chain Reaction (“RT-PCR”). In certain non-limiting embodiments, the expression of at least one stemness marker is measured by a method comprising quantitative RT-PCR (qRT-PCR). In certain embodiments, the stemness marker is detected by a method comprising RT-PCR or qRT-PCR using one or more pairs of probes.

In certain non-limiting embodiments, the stemness marker is detected by antibody binding analysis such as, but not limited to, Western Blot analysis and immunohistochemistry.

In certain non-limiting embodiment, the stemness stability comprises determining the expression of at least one stemness marker selected from the group consisting of ATP-binding cassette super-family G member 2 (ABCG2), neuroepithelial stem cell protein (NESTIN), tumor protein p63 (isoform ANp63), paired box protein 6 (Pax6), Bmi-1, SSEA-1, SSEA-4, TRA1-60, TRO1-81, alkaline phosphatase, Fzd-1, Oct3/4, Sox2, Sox3, Sox9, Sox10, Klf-2, Klf-4, Klf-5, C-MYC, NGFR, and NANOG.

In certain non-limiting embodiments, present disclosure includes determining a ratio of inflammation (RInflam) of the CSSC sample. In certain non-limiting embodiments, the ratio of inflammation is a determination of the anti-inflammatory potency of the CSSC sample. In certain non-limiting embodiments, the anti-inflammatory potency of the CSSC sample is determined by measuring the CSSC sample's ability to modulate the inflammatory milieu of a cell model. In certain non-limiting embodiments, the CSSC sample's ability to modulate the inflammatory milieu of a cell model includes culturing the cell model in the presence of conditioned media obtained from a CSSC culture and determining the expression level of at least one gene modulated by an inflammatory response.

In a non-limiting example, the RInflam of a CSSC sample is calculated as described in the below Example. In a certain embodiment, RInflam can be defined as a quantification of gene expression in a cell model in response to a CSSC sample or conditioned media obtained from a CSSC sample. In certain embodiments, the quantification of gene expression can be represented as a Cycle Threshold (CT) value, gene fold change or delta CT value, as determined by known PCR-based methods. In certain embodiments, gene expression is determined in a cell model having received treatment with conditioned media obtained from a CSSC sample culture. In certain embodiments, the gene expression of a cell model treated with conditioned media obtained from a CSSC sample culture (referred to as “native”) is compared to the gene expression of a control cell model treated with heat-denatured CSSC (referred to as “denatured”). In certain embodiments, the RInflam is a measure of the sum of gene expression fold change after treatment of native versus denatured conditions. In certain embodiments, the RInflam serves as a measure of the CSSC sample's ability to modulate the inflammatory environment, indicating its anti-inflammatory potency.

In certain non-limiting embodiments, the cell model is an immortalized cell line or primary cell culture. In certain non-limiting embodiments, the cell model is an inflammatory cell model. In certain non-limiting embodiments, the cell model is of the hematopoietic cell lineage. In certain non-limiting embodiments, the cell model undergoes macrophage-osteoclastogenesis differentiation. In certain non-limiting embodiment, the cell model undergoes macrophage-osteoclastogenesis differentiation.

In certain non-limiting embodiments, the cell model is an immortalized cell line. In certain non-limiting embodiment, the cell line is a hemopoietic progenitor cell line of the monocyte-macrophage lineage. In certain non-limiting embodiments, the cell line is a mouse macrophage line, RAW-264.7.

In certain non-limiting embodiments, the cell model is a primary cell line derived from hematopoietic progenitors. In certain non-limiting embodiments, the hematopoietic progenitors are obtained from bone marrow tissue. In certain non-limiting embodiments, the hematopoietic progenitors can be obtained from bone marrow tissue from a human, bovine, mouse or rat.

In certain non-limiting embodiments, the present disclosure includes determining the gene expression of cell model in response to treatment with conditioned media obtained from a CSSC sample. In certain embodiment, gene expression is determined for at least one gene modulated by an inflammatory response. In certain embodiments the at least one gene modulated by an inflammatory response is gene involved in macrophage-osteoclastogenesis differentiation. In certain embodiments, the at least one gene involved in macrophage-osteoclastogenesis differentiation is selected from a group consisting of tartrate-resistant acid phosphatase (TRAP/ACP5), matrix metalloproteinase 9 (MMP9), cathepsin K (CTSK), calcitonin receptor (CTR), carbonic anhydrase II (CAII), and a combination thereof.

In certain non-limiting embodiments, a CSSC sample's ability to modulate an inflammatory response includes inhibition or suppression of at least one gene modulated by an inflammatory response. In certain non-limiting embodiment, CSSC sample's ability to inhibit or suppress at least one gene modulated by an inflammatory response is a positive indicator of anti-scarring capability. In a non-limiting example, the SI score of a CSSC sample is calculated as described in the Example below. In certain non-limiting embodiments, the present disclosure includes determining a scarring index (SI) score of the CSSC sample, wherein the SI score is representative of the effectiveness of the CSSC sample for reduction of corneal scar formation in a CSSC therapy. In certain embodiments, the SI score of the CSSC sample is based on the expression of at least one stemness marker in the CSSC in sample and the RInflam value of the CSSC sample, i.e., sum of gene expression fold change ratio after treatment of native versus denatured conditions. In certain embodiments, a SI value of less than 10 predicts the CSSC sample to provide approximately 50% scar reduction. In certain embodiments, a SI value of less than 10 predicts the CSSC sample to be ineffective in scar inhibition/suppression.

In certain non-limiting embodiments, the present disclosure includes testing the in vivo anti-scarring effectiveness of a CSSC sample using a mouse model of anterior corneal stromal injury. In certain non-limiting embodiments, the mouse model of anterior corneal stromal injury includes creating an anterior stromal wound and immediately after injury applying a CSSC sample in a fibrin gel to the injured corneal surface, followed by topical antibiotics to prevent infection. In certain non-limiting embodiments, the injury scar area and overall corneal area are quantified by imaging analysis software and mean % scarring is calculated.

### Methods of Treatment

In one embodiment, the present disclosure further provides for methods for treating, reducing, or preventing corneal scarring in a subject in need thereof. The methods include obtaining a corneal stromal stem cell (CSSC) sample; determining a scarring index (SI) score of the CSSC sample, wherein an SI score is representative of the effectiveness of the CSSC sample as a therapy for treating, reducing, or preventing corneal scar formation in a subject; formulating an anti-scarring corneal scar therapy comprising the CSSC sample, and administering to the subject a therapeutically effective amount of anti-scarring corneal therapy to a target corneal tissue in the subject.

In certain embodiments, the subject can be treated with the above-described methods for eye-related diseases/disorders and ocular repair/wound healing associated with compromised corneal transparency, corneal scar formation, secondary cataract formation, glaucoma filtration surgery, ocular surgical procedures and implants, photorefractive keratectomy, laser in situ keratomileusis, formation and contraction of pre- and epiretinal membranes, proliferative vitreoretinopathy, proliferative diabetic retinopathy, diabetic macular edema, subretinal fibrosis/scarring, retinal gliosis, and formation of choroidal membranes, age-related macular degeneration, and retinal vein occlusion.

In some embodiments, the anti-scarring therapy can be administered to a target corneal tissue by any method known in the art, including, but not limited to, topical instillation, periocular injection, intravitreal injection, systemic administration, or the insertion of a reservoir that provides sustained release of the anti-scarring therapy. In certain embodiments, the CSSC sample can be formulated in a form, wherein the form can be selected from the group consisting of a solution, a suspension, a semi-solid gel, a gel, an emulsion, semi-liquid, an ointment, a cream, foam gel, a controlled-release/sustain-release vehicle, an eye drop, and combinations thereof. In certain embodiments, administration comprises applying the anti-scarring therapy to the cornea or the site of injury, wound or defect for a time period sufficient to reduce the injury, wound or defect.

### EXAMPLE

The presently disclosed subject matter will be better understood by reference to the following Examples, which are provided as exemplary of the presently disclosed subject matter, and not by way of limitation.

The present Example is directed to the determination that variation in CSSC effectiveness is affected by stem cell stability and other measurable cell features. The present methods demonstrate a clarification of the most relevant indicators to establish quality control standards to identify CSSC with good healing/regenerative capability and anti-scarring effect, or to screen out CSSC with poor anti-scarring activity that should be excluded from treatments.

### 1. Stem Cell Stability—Expression of Stemness Markers ABCG2 and NESTIN

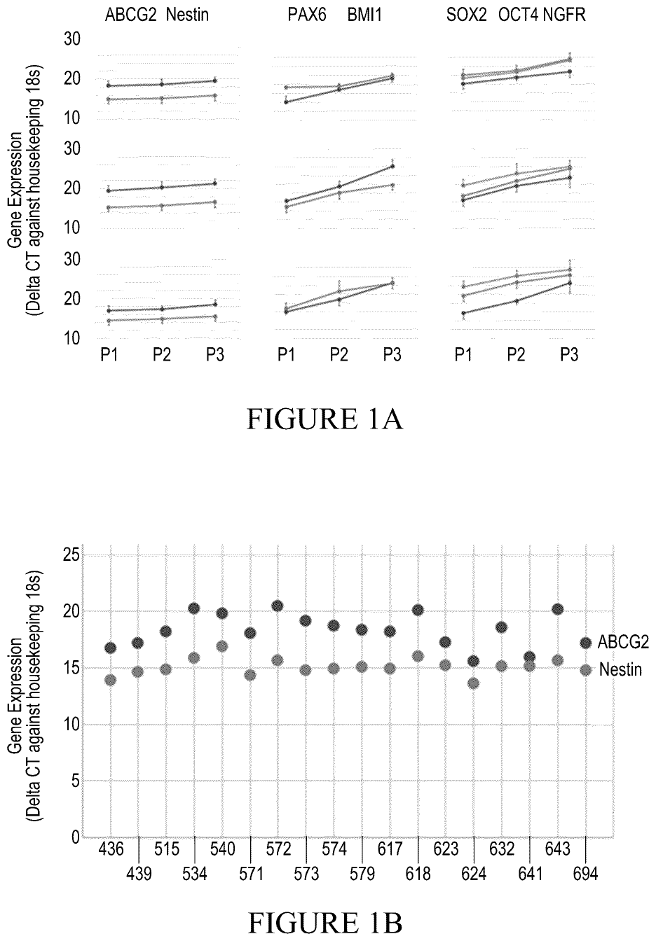

Human CSSC batches at passage 2 to 3 were collected for total RNA extraction, and the purified RNA samples were assayed by quantitative polymerase chain reaction for the expression of various stem cell markers, including human ABCG2, NESTIN, Pax6, Bmi-1, Sox2, Oct4, and NGFR, with housekeeping 18S for normalization, using validated primers. Relative RNA abundance (ΔCT) of each stem cell marker was normalized to 18S (FIG. 1A). In studying the stemness gene expression regarding cell passages, there were two expression patterns: ABCG2 and NESTIN were consistently expressed at P1, P2 and P3, but the expression of Pax6, Bmi-1, Sox2, Oct4 and NGFR were gradually downregulated (FIG. 1A). At P2, ABCG2 and NESTIN showed a consistent trend of expression. The ACT of ABCG2 (normalized with housekeeping 18S) was about 16 to 20, whereas that of NESTIN was 12 to 16 (FIG. 1B). This indicated that primary CSSC had a stronger expression of NESTIN than ABCG2, and the difference was maintained within 3 to 5 ACT values.

### 2. Anti-Inflammatory Potency—Inhibition Efficiency on a Chronic Pro-Inflammatory Macrophage-Osteoclastogenesis

CSSC with high regenerative potential effectively inhibit the differentiation of a mouse macrophage line, RAW264.7 cells (ATCC TIB-71), towards a pro-inflammatory osteoclast lineage. Conditioned media (CM) obtained from human CSSC cultures was concentrated to ˜ 1/20^{th} volume and total protein was quantified by BCA method (Bicinchroninic acid assay or Smith assay). Half of the sample was heat-denatured and half was kept as native. RAW cells were pre-treated with naïve or heat-denatured CM concentrate (CMconc, 500 μg protein) for 30 min, then RANKL peptide (50 ng/ml) and ConA (20 μg/ml) were added to induce osteoclast differentiation.

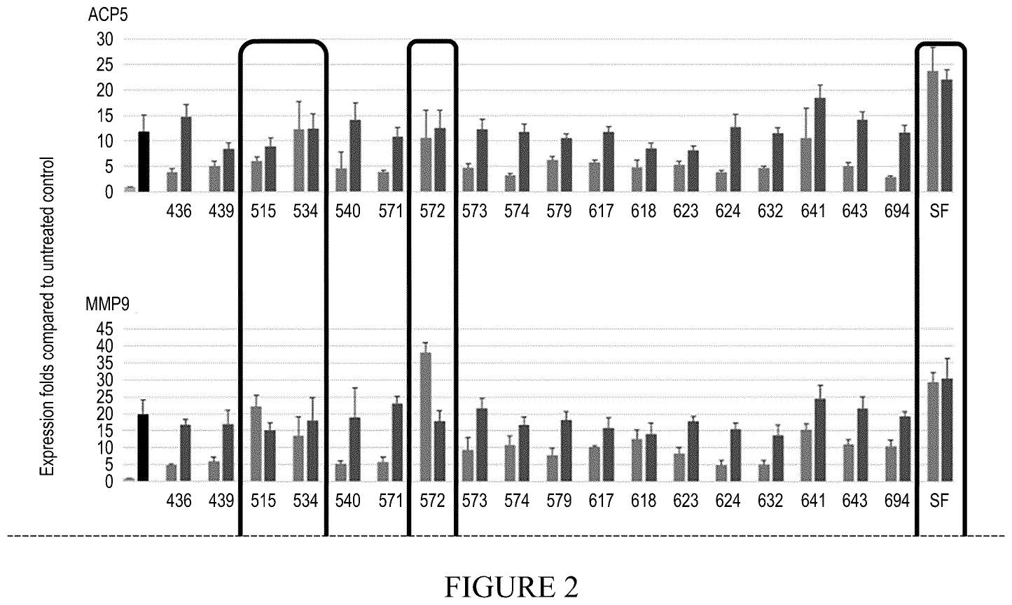

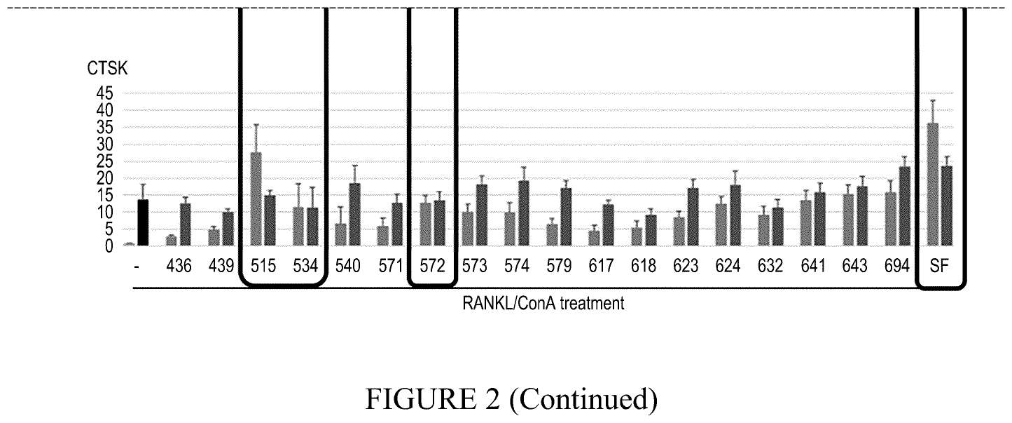

After 5 days, total cellular RNA was extracted for expression analysis of osteoclast specific genes (alkaline phosphatase 5 [ACP5], matrix metalloproteinase 9 [MMP9], and cathepsin K [CTSK]). The gene expression fold change was calculated for both naïve and heat-denatured CMconc treatments by comparing against the control RAW cells. The treatments with native CMconc from most CSSC, including HC436, 439, 540 and 641, consistently downregulated all 3 genes, when compared with the corresponding heat-denatured CMconc treatments. On the other hand, native CMconc from HC515, 534, and 572 did not suppress all 3 genes relative to the denatured CMconc treatments (brackets in FIG. 2, including HCF corneal fibroblasts as negative control).

The ratio of inflammation (RInflam) effected by the treatment of native versus denatured CM was calculated as the sum of expression fold change ratio of all 3 genes.

$$\sum RInflamACP 5 + MMP 9 + CTSK = ACP 5 ( naïve / denatured ) + MMP 9 ( naïve / denatured ) + CTSK ( naïve / denatured )$$

Lower RInflam values represent CSSC with lower inflammatory outcome.

### 3. Evaluation of Stem Cell Stability and Anti-Inflammatory Capability to Indicate In Vivo Anti-Scarring Potency

A novel formula integrating ΔCT (ABCG2), ΔCT (NESTIN) and RInflam of osteoclast gene expression to calculate Scarring Index representing the scarring potency for CSSC.

$$Scarring Index ( S I ) = [ {2}^{Δ CT ( ABCG 2 )} + {2}^{Δ CT ( NES )} ] / m + {2}^{\sum RInflam} / n \\ ( m and n represent constants )$$

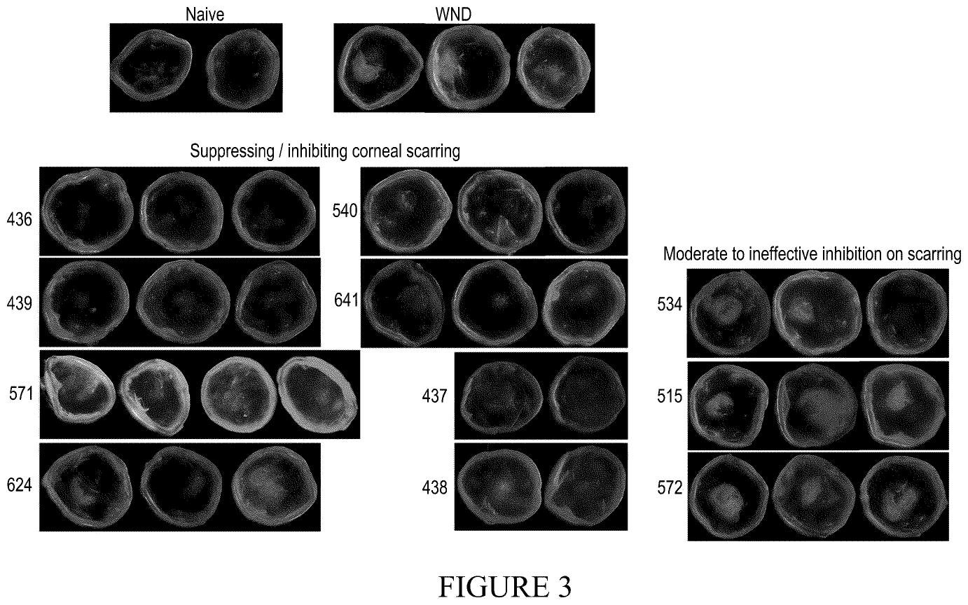

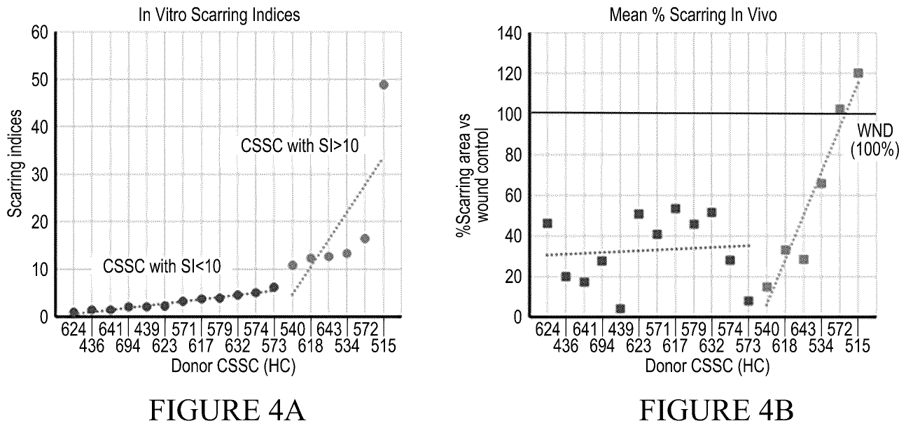

The Scarring indices of 18 CSSC batches from different donor corneas were calculated (FIG. 4C). These 18 CSSC batches were also tested for their in vivo anti-scarring effectiveness using a mouse model of anterior corneal stromal injury. Anterior stromal wound (˜0.5 mm diameter, 10-20 μm depth) was created by Algerbrush ablation [6-11]. Immediately after injury, CSSC (5×10⁴ cells) in a drop of fibrin gel (1 μl volume) were applied on the injured corneal surface. Tobramycin (antibiotics) eye drops were applied for the subsequent 3 days, thrice daily. Each cell treatment was conducted on 8 corneas. At day 14, the mouse corneas were harvested for corneal scar evaluation. Visual assessment showed that naïve corneas were clear and untreated wound corneas had intense scarring (FIG. 3). Corneas treated with different batches of CSSC resulted in different degrees of scar inhibition. As an example, HC436, 439, 540, 641 showed good anti-scarring effect. In contrast, HC515 and 572 were ineffective to prevent scarring.

The scar area and overall corneal area were quantified by ImageJ software and the mean % scarring was calculated for the group of corneas treated with same CSSC batch (FIG. 4B). The scarring indices (SI) of each CSSC batch was also calculated by the invented formula and the values for each CSSC batch were shown in the same order as for the respective treatment outcome (% scarring). FIG. 4A plotted the SI calculated for 18 different donor CSSC and the calculation details are listed in Table 1. Twelve of them had SI values <10 and the remaining six had higher SI. All these CSSC batches at P3 were tested in vivo for their anti-scarring effects and the treated corneas presented different scar inhibitory outcomes. As an example, HC436, 439, and 641 treatments reduced scar formation, whereas HC515, 534, and 572 were moderate to ineffective in inhibiting corneal scarring (FIG. 3). The overall results of corneal wound treatment (% scarring values) by 18 different CSSC batches are listed in Table 1.

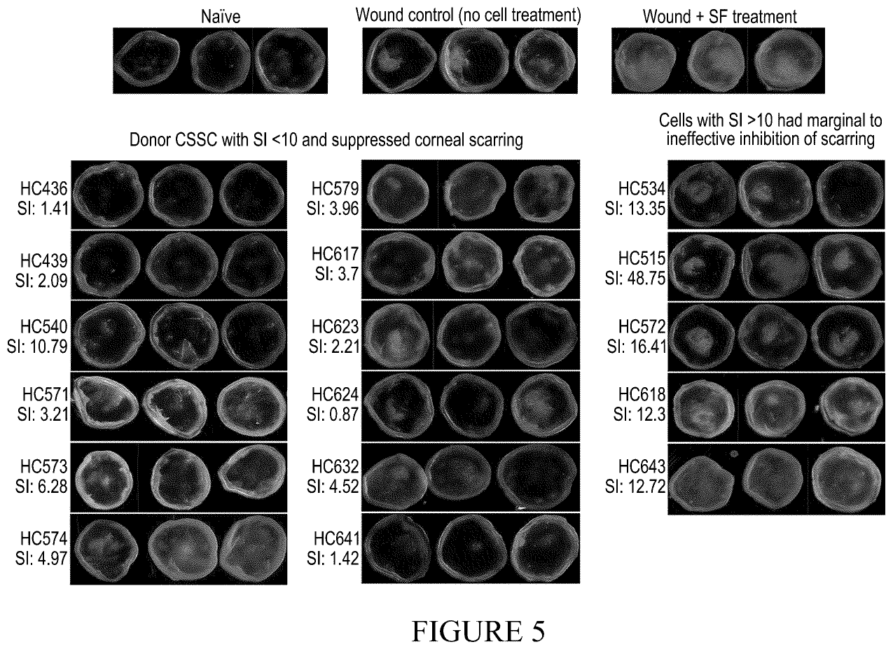

When this scarring outcome was correlated to the SI values, the cells with SI<10 (blue round dots in FIG. 4A) had about 50% or less scar area (blue squares in FIG. 4B), compared to wound controls (dark horizontal line in FIG. 4B). The linear regression line (blue dotted line) showed a stable outcome of 32.7±17.4% scar inhibition. On the other hand, treatment with cells having SI>10 (orange round dots in FIG. 4A) showed moderate to ineffective (orange squares in FIG. 4B), though HC540, 618, and 643 that had SI marginally more than 10 show an opacity clearance. The orange linear regression line exhibited an increasing trend of scar formation. The treatment outcomes by these 2 groups of cells: SI<10 versus SI>10 showed a statistically significant difference (P=0.0312, Wilconxon Signed Rank test). The overall result of corneal wound treatment with 18 different CSSC batches accompanied with their SI valves is shown in FIG. 5.

TABLE 1
Calculation of in vitro scarring indices (SI) of CSSC and comparison to the respective
in vivo percentages of scarring area after cell treatment to mouse corneas.

| CSSC | | | 2^{ΔCT(ABCG2)} + | | | | | In vitro | |
|---|---|---|---|---|---|---|---|---|---|
| at | ΔCT | ΔCT | 2^{ΔCT(NES)}/ | | | | | Scarring | In vivo |
| P2 | (ABCG2) | (NES) | 100,000 | ACP5 | MMP9 | CTSK | 2^{ΣRInflam}/10 | indices | % Scarring |
| 436 | 16.72 ± 0.28 | 13.9 ± 0.35 | 1.232 | 0.26 | 0.30 | 0.24 | 0.174 | 1.406 | 19.9 ± 16.6 |
| 439 | 17.21 ± 0.32 | 14.65 ± 0.41 | 1.773 | 0.60 | 0.61 | 0.43 | 0.312 | 2.085 | 4.2 ± 1.3 |
| 515 | 18.17 ± 0.21 | 14.83 ± 0.07 | 3.241 | 0.78 | 3.44 | 4.61 | 45.509 | 48.750 | 119.9 ± 55.5 |
| 534 | 20.21 ± 0.33 | 15.86 ± 0.15 | 12.724 | 1.00 | 0.64 | 1.01 | 0.628 | 13.352 | 65.5 ± 52.8 |
| 540 | 19.83 ± 0.28 | 16.86 ± 0.14 | 10.510 | 0.42 | 0.59 | 0.46 | 0.277 | 10.787 | 14.9 ± 6.4 |
| 571 | 18.09 ± 0.17 | 14.32 ± 0.29 | 2.995 | 0.36 | 0.25 | 0.46 | 0.210 | 3.205 | 40.5 ± 39.2 |
| 572 | 20.47 ± 0.31 | 15.66 ± 0.34 | 15.042 | 0.77 | 2.07 | 0.93 | 1.364 | 16.406 | 102.3 ± 14.1 |
| 573 | 19.13 ± 0.34 | 14.8 ± 0.11 | 6.023 | 0.38 | 0.44 | 0.56 | 0.260 | 6.283 | 8.1 ± 9.2 |
| 574 | 18.74 ± 0.26 | 14.96 ± 0.06 | 4.697 | 0.28 | 0.65 | 0.52 | 0.273 | 4.970 | 28 ± 19.2 |
| 579 | 18.36 ± 0.15 | 15.08 ± 0.15 | 3.711 | 0.59 | 0.43 | 0.38 | 0.264 | 3.975 | 45.5 ± 28.8 |
| 617 | 18.24 ± 0.26 | 14.93 ± 0.23 | 3.408 | 0.49 | 0.65 | 0.38 | 0.287 | 3.695 | 53.2 ± 18.5 |
| 618 | 20.1 ± 0.34 | 15.99 ± 0.11 | 11.889 | 0.56 | 0.90 | 0.59 | 0.414 | 12.303 | 33.1 ± 17.1 |
| 623 | 17.22 ± 0.2 | 15.19 ± 0.15 | 1.900 | 0.65 | 0.47 | 0.5 | 0.307 | 2.207 | 50.7 ± 51.9 |
| 624 | 15.61 ± 0.39 | 13.59 ± 0.21 | 0.623 | 0.30 | 0.32 | 0.69 | 0.248 | 0.871 | 45.9 ± 34.6 |
| 632 | 18.56 ± 0.35 | 15.11 ± 0.29 | 4.218 | 0.41 | 0.37 | 0.8 | 0.299 | 4.517 | 51.4 ± 44.9 |
| 641 | 15.93 ± 0.26 | 15.12 ± 0.09 | 0.980 | 0.66 | 0.65 | 0.81 | 0.435 | 1.415 | 17.2 ± 9.4 |
| 643 | 20.18 ± 0.49 | 15.63 ± 0.2 | 12.386 | 0.35 | 0.51 | 0.87 | 0.332 | 12.718 | 28.3 ± 31.2 |
| 694 | 17.21 ± 0.23 | 14.79 ± 0.17 | 1.799 | 0.25 | 0.54 | 0.68 | 0.277 | 2.076 | 27.6 ± 8.7 |
| HCF | | | | 1.12 | 0.98 | 1.93 | 1.634 | | |

Two QC parameters - ΔCT of ABCG2 and NESTIN (mean ± SD, normalized with the housekeeping 18S) of donor CSSC, and rate of inflammation (expression fold changes of ACP5, MMP9, and CTSK after treatment of native versus denatured CSSC-derived CMconc in RAW 267.4 cells.
Scarring index was calculated as the sum of 2^{ΔCT(ABCG2)} + 2^{ΔCT(NES)}/100.000 + 2^{ΣRInflam}/10.
A comparison to HCF (human corneal stromal fibroblasts) with expression fold changes of ACP5, MMP9, and CTSK was included in the osteoclastogenesis assay.

### 4. Conclusion

The method provided here demonstrate the development of a new formula integrating stem cell stability and anti-inflammatory capability of human CSSC to calculate the Scarring Index, representing effectiveness of CSSC in prevention or reduction of corneal scarring. This calculation can predict the anti-scarring potential of CSSC when used for cell therapy in patients with corneal scarring diseases. Cells with SI<10 are anticipated to have ˜50% scar reduction. SI>10 are predicted to be ineffective in scar inhibition/suppression and the cells should be excluded for treatment use.
Cell exclusion criteria—SI>10
Cell inclusion criteria—SI<10 to anticipate ˜50% scar reduction

### REFERENCES

1 Whitcher, J. P., Srinivasan, M. & Upadhyay, M. P. Corneal blindness: a global perspective. Bulletin of the World Health Organization 79, 214-221 (2001).
2 Du, Y., Funderburgh, M. L., Mann, M. M., SundarRaj, N. & Funderburgh, J. L. Multipotent stem cells in human corneal stroma. Stem cells 23, 1266-1275, doi: 10.1634/stemcells.2004-0256 (2005).
3 Du, Y. et al. Stem cell therapy restores transparency to defective murine corneas. Stem cells 27, 1635-1642, doi: 10.1002/stem.91 (2009).
4 Funderburgh, J. L., Funderburgh, M. L. & Du, Y. Stem Cells in the Limbal Stroma. The ocular surface 14, 113-120, doi: 10.1016/j.jtos.2015.12.006 (2016).
5 Hertsenberg, A. J. et al. Corneal stromal stem cells reduce corneal scarring by mediating neutrophil infiltration after wounding. PloS One 12, e0171712, doi: 10.1371/journal.pone.0171712 (2017).
6 Basu, S., Damala, M., Tavakkoli, F., Mitragotri, N. & Singh, V. Human Limbus-derived Mesenchymal/Stromal Stem Cell Therapy for Superficial Corneal Pathologies: Two-Year Outcomes. Invest Ophthalmol Vis Sci 60, 4146 (2019).
7. Khandaker, I. et al. A novel transgenic mouse model for corneal scar visualization. Exp Eye Res 200, 108270, doi: 10.1016/j.exer.2020.108270 (2020).
8 Weng, L. et al. The anti-scarring effect of corneal stromal stem cell therapy is mediated by transforming growth factor b3. Eye Vis) 7, 52, doi: 10.1186/s40662-020-00217-z (2020).
9 Basu, S., Damala, M., Tavakkoli, F., Mitragotri, N. & Singh, V. Human Limbus-derived Mesenchymal/Stromal Stem Cell Therapy for Superficial Corneal Pathologies: Two-Year Outcomes. Invest Ophthalmol Vis Sci 60, 4146 (2019).
10 Jhanji, V, Santra M, Riau A K, Geary M L, Yang T, Rubin E, Yusoff N Z, Dhaliwal D K, Mehta J S, Yam G H. Combined Therapy Using Human Corneal Stromal Stem Cells and Quiescent Keratocytes to Prevent Corneal Scarring after Injury. Int. J. Mol. Sci. 23 (13), 6980; doi.org/10.3390/ijms23136980. (2022)
11. Yam G H F, Yang T B, Geary M L, Funderburgh M L, Santra M, Rubin E, Du Y, Sahel J A, Jhanji V, Funderburgh J L. Human corneal stromal stem cells express anti-fibrotic microRNA-29a and 381—5p-a robust cell selection tool for stem cell therapy of corneal scarring. J Adv Res. S2090-1232 (22) 00120-5 (2022).

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

What is claimed is:

1. A method of determining effectiveness of corneal stromal stem cell (CSSC) therapy, the method comprising:
(a) obtaining a CSSC sample;
(b) measuring expression of at least one stemness marker in the CSSC sample for stem cell stability (ΔCT);
(c) creating a conditioned medium by culturing the CSSC sample until the cell culture medium contains the desired level of extracellular product;
(d) introducing the CSSC sample conditioned medium into a cell-based model of inflammation;
(e) measuring expression of at least one biomarker in the cell-based model (ΔCT);
(f) determining a ratio of inflammation of the CSSC sample (RInflam), wherein the RInflam is based on the expression fold change of at least one biomarker in the cell-based model; and
(g) determining a scarring index (SI) score of the CSSC sample, where the SI is based on the expression of at least one stemness marker in the CSSC in (c) and the expression of at least one biomarker in the cell-based model in (e), wherein an SI score is representative of the effectiveness of the CSSC sample for reduction of corneal scar formation in CSSC therapy.

2. The method of claim 1, wherein the at least one stemness marker is selected from the group consisting of ABCG2, Nestin, and combinations thereof.

3. The method of claim 1, wherein the ratio of inflammation is determined by the treatment of native versus denatured CSSC conditioned media in a cell-based model of inflammation, and calculating a sum of expression fold change ratio of targeted osteoclast gene.

4. The method of claim 3, wherein the targeted osteoclast genes comprise alkaline phosphatase 5 [ACP5], matrix metalloproteinase 9 [MMP9], and cathepsin K [CTSK], and the ratio of inflammation is calculated according to:
$$\sum {RInflam}_{ACP 5 + MMP 9 + CTSK} = ACP 5 ( naïve / denatured ) + MMP 9 ( naïve / denatured ) + CTSK ( naïve / denatured ) .$$

5. The method of claim 1, wherein the scarring index score is calculated according to:
$$Scarring Index ( S I ) = [ {2}^{Δ CT ( ABCG 2 )} + {2}^{Δ CT ( NES )} ] / m + {2}^{\sum RInflam} / n$$
wherein m and n represent constants.

6. The method of claim 1, wherein an SI score of less than 10 represents effectiveness of the CSSC sample for reduction of corneal scar formation in CSSC therapy.

7. The method of claim 6, wherein reduction of corneal scar formation is about 50%.

8. The method of claim 1, wherein an SI score greater than 10 represents ineffectiveness of the CSSC sample for reduction of corneal scar formation in CSSC therapy.

9. The method of claim 3, wherein the expression of at least one stemness marker or osteoclast gene is determined by reverse transcription polymerase chain reaction.

10. The method of claim 1, wherein the cell-based model of inflammation comprises a cell of the hematopoietic lineage capable of undergoing osteoclastogenesis.

11. The method of claim 10, wherein the cell-based model comprises a macrophage cell.

12. The method of claim 11, wherein the macrophage cell comprises a RAW264.7 cell or derivative thereof.

13. The method of claim 12, wherein the RAW cell or derivative thereof is induced to undergo osteoclast differentiation.

14. The method of claim 13, wherein the RAW cell is pre-treated with native or heat-denatured conditioned media prior to induction of osteoclast differentiation.

15. The method of claim 1, wherein the effectiveness of CSSC therapy is determined in an in vivo model of corneal stromal injury.

16. The method of claim 15, wherein the in vivo model of corneal injury is a murine mouse model of anterior corneal stromal injury.

17. A method of treating, reducing, or preventing corneal scarring in a subject in need thereof, the method comprising:
(a) obtaining a corneal stromal stem cell (CSSC) sample;
(b) determining a scarring index (SI) score of the CSSC sample, wherein an SI score is representative of the effectiveness of the CSSC sample as a therapy for treating, reducing or preventing of corneal scar formation in CSSC therapy;
(c) formulating an anti-scarring therapy comprising the CSSC sample; and
(d) administering a therapeutically effective amount of the anti-scarring therapy to a target corneal tissue in the subject.
